(19) United States
(12) Patent Application Publication
(10) Pub. No.: US 2026/0308095 A1
(43) Pub. Date: Oct. 8, 2026

(54) PROCESS FOR PRODUCING A CANNABIS PRODUCT

(71) Applicant: Mark Zugsmith, Eugene, OR (US)

(72) Inventor: Mark Zugsmith, Eugene, OR (US)

(21) Appl. No.: 19/173,035

(22) Filed: Apr. 8, 2025

(51) Int. Cl. C07C 37/00; C07D 311/80

(52) U.S. Cl. CPC C07C 37/004; C07D 311/80

(57) ABSTRACT

A process for producing a cannabis product. The process includes collecting a cannabis biomass having certain desirable qualities and washing the cannabis biomass to create a washed cannabis biomass. The process also includes the steps of wet grinding the washed cannabis biomass to create a wet-ground cannabis biomass; and drying the wet-ground cannabis biomass to create a dried cannabis biomass.

### STATEMENT REGARDING FEDERALLY SPONSORED RESEARCH OR DEVELOPMENT

Not applicable.

### CROSS-REFERENCE TO RELATED APPLICATIONS

Not applicable.

### BACKGROUND OF THE DISCLOSURE

### 1. Field of the Invention

The present disclosure relates to a process for producing a cannabis product having certain beneficial attributes.

### 2. Description of the Related Art

There are various methods of processing cannabis to generate cannabis products having certain properties. A lot of methods for processing cannabis separate the oil from the plant material. Additionally, various cannabis products cause anxiety and/or stress for the users.

Accordingly, there is a need for a process that produces a cannabis product that reduces anxiety and stress.

### SUMMARY OF THE DISCLOSURE

The present disclosure is directed to a process for producing a cannabis product. The process includes collecting a cannabis biomass having certain desirable qualities and washing the cannabis biomass to create a washed cannabis biomass. The process also includes the steps of wet grinding the washed cannabis biomass to create a wet-ground cannabis biomass; and drying the wet-ground cannabis biomass to create a dried cannabis biomass.

### DETAILED DESCRIPTION OF THE DISCLOSURE

The present disclosure is directed to a process for producing a cannabis product having certain desirable characteristics made from a cannabis biomass without the use of a solvent. There are numerous strains of cannabis and combinations thereof that can be used as a cannabis biomass for producing the cannabis product described herein. The process can include starting with the cannabis biomass having certain qualities, such as a certain tetrahydrocannabinol (THC) content by weight of the total cannabinoid of the cannabis biomass. In one embodiment, the THC content of the total cannabinoid of the cannabis biomass is about 5% to about 50%. In another embodiment, the THC content of the total cannabinoid of the cannabis biomass is about 10% to about 40%. In a further embodiment, the THC content of the total cannabinoid of the cannabis biomass is about 14% to about 30%.

The cannabis biomass can be provided in any form known for cannabis. For example, the cannabis biomass can be provided as full flowers, b-buds (littles) and/or sugar trim. The cannabis biomass for the process can also have a certain ratio of cannabidiol (CBD) to THC. In one embodiment, the ratio of CBD to THC is from about 1:1 to about 7:1. In another embodiment, the ratio of CBD to THC is from about 3:1 to about 6:1. In a further embodiment, the ratio of CBD to THC is from about 4.5:1 to about 5.5:1. In yet another embodiment, the ratio of CBD to THC is about 5:1.

The cannabis biomass can be subjected to a washing and sublimation step of the process to generate a washed cannabis biomass. In one embodiment of the washing and sublimation step, the cannabis biomass can be soaked in tepid or lukewarm water for a certain amount of time, such as for 5-20 minutes or for 12-15 minutes. The water can then be drained and the cannabis biomass can be soaked again in tepid or lukewarm water for a certain amount of time, such as for 5-20 minutes or for 12-15 minutes. Again, the water can be drained and fresh tepid or lukewarm water can be combined with the cannabis biomass and heated for a certain amount of time. The cannabis biomass can be heated to a range of about 185 degrees Fahrenheit to about 205 degrees Fahrenheit for about 10-20 minutes, or it could be heated for about 15-18 minutes. The water could then be drained leaving the washed cannabis biomass for further processing.

In another embodiment of the washing and sublimation step, the washed cannabis biomass can be generated by soaking the cannabis biomass in tepid or lukewarm water for a certain amount of time, such as for 5-20 minutes or for 12-15 minutes. The water can then be drained, and the cannabis biomass can be soaked again in tepid or lukewarm water for a certain amount of time, such as for 5-20 minutes or for 12-15 minutes. After draining the water from the cannabis biomass, the cannabis biomass can be heated under a certain pressure for a certain amount of time. The pressure the cannabis biomass can be heated under ranges from about 1 psi to about 15 psi above atmospheric pressure, or in the alternative about 10 to about 15 psi above atmospheric pressure. The temperature the cannabis biomass can be heated while under pressure is in a range of about 215 degrees Fahrenheit to about 240 degrees Fahrenheit. The cannabis biomass can be heated under pressure for about for about 5 -15 minutes, or in the alternative for about 7-10 minutes from the time steam has collected, or from the time the cannabis biomass is at the desired pressure.

In a further embodiment of the present disclosure, the cannabis biomass can be loaded into a steam distillation apparatus where water is boiled below the cannabis biomass and the steam generated from the boiling water passes up and through the cannabis biomass. In another embodiment, the cannabis biomass could be run through a hydro distillation process where the cannabis biomass is in the water that is boiled. The cannabis biomass can be brought to a slow boil by bringing the temperature to about 100 degrees Celsius to about 105 degrees Celsius. Once the first drops of essential oil (terpenes) are created, the cannabis biomass can be heated for about 30 minutes to about an hour and fifteen minutes, or alternatively, for about 40 minutes to about an hour. Or, the cannabis biomass is heated until the distillation/separation of essential oil (terpenes) is complete, which can be determined by when the smell of the process turns from a floral smell to a grassy smell. In this embodiment, the essential oil generated from the distillation apparatus can be allowed to breathe for around 12-24 hours and used for various purposes. The distillation apparatus can be made of any material known in the art. In one embodiment, the distillation apparatus can be made of copper which contributes to giving the cannabis product certain desirable characteristics.

The process of creating the biomass product can also include a black pepper processing step. In this step a certain amount of whole black peppercorns can be soaked in water for a certain amount of time to create washed black peppercorns. In one embodiment, the black peppercorns could be soaked in water for about 30 minutes to about 90 minutes. A certain amount of black peppercorns can be used relative to the amount of cannabis biomass that is used to create the cannabis product. In one embodiment, the weight of black peppercorns used is about 2% to about 10% of the weight of the cannabis biomass used. In another embodiment, the weight of black peppercorns used is about 3% to about 8% of the weight of the cannabis biomass used.

After the washing and sublimation step, the washed cannabis biomass can be subjected to a wet grinding step that creates a wet-ground cannabis biomass. Prior to the wet grinding step, the water can be removed from the cannabis biomass and the black peppercorns, but they are not dried. The washed cannabis biomass and the washed black peppercorns can be ground separately and then combined, or they can be combined prior to the wet grinding step. The washed cannabis biomass and the washed black peppercorns can be ground in one large batch at once or they could be ground by adding a little more of the washed cannabis biomass and the washed black peppercorns throughout the grinding step. The washed cannabis biomass and the washed black peppercorns can be ground via any type of grinder known in the art such that the washed cannabis biomass and the washed black peppercorns can be ground into a smooth paste. The grinder could be a small handheld grinder all the way up to giant commercial grinders. The washed cannabis biomass and the washed black peppercorns can be ground

The wet-ground cannabis biomass can be dried in the next step of the process to create a dried cannabis biomass. The wet-ground cannabis biomass can be dried via any process known in the art. The wet-ground cannabis biomass can be laid out to air dry. They wet-ground cannabis biomass can be rolled into ball-like shaped and wrapped in absorbent materials and air dried. In another embodiment, the wet-ground cannabis biomass could be subject to a dehydration process using an oven or a dehydrator apparatus. The dehydration process includes subjecting the wet-ground cannabis biomass to a certain temperature for a certain amount of time to achieve a desired dryness. The temperature for the dehydration process can be between about 122 degrees Fahrenheit and about 150 degrees Fahrenheit.

After the dried cannabis biomass has been produced via the drying step, the dried cannabis biomass can then be subjected to a dry grinding process to create the cannabis product. The dried cannabis biomass can be ground via any grinding apparatus known in the art. The dried cannabis biomass can be ground in small batches by handheld device, or the dried cannabis biomass can be ground with commercial grinders. In one embodiment, the dried cannabis biomass is ground in small increments of time to prevent the cannabis product from getting too hot. If the cannabis product gets too hot it will become sticky and begin binding to itself. In one embodiment, the dried cannabis biomass is ground for less than 45 seconds at a time. In another embodiment, the dried cannabis biomass is ground for less than 30 seconds at a time. In yet another embodiment, the dried cannabis biomass is ground for less than 20 seconds at a time. The dried cannabis biomass is ground until the cannabis product is in a powder form and the particles are less than a particular size. The cannabis product can be run through a separation process to allow only the desired size particles to be captured. The separation process can include any apparatus/device capable of separating the desired sized particles from the other particles, such as a sieve, series of sieves, vibratory sieve, or any other type of sieve product. The particles that are not small enough can be sent through the dry grinding process again. The desired particle size is between about 53 microns to about 125 microns.

The cannabis product with the desired sized particles can then be stored in any type of container with some type of moisture control or moisture removal device. In one embodiment, the cannabis product can be stored in a sealed container with nitrogen. The product can also be stored in a cool, dark place.

From the above description, it is clear that the present disclosure is well-adapted to carry out the objectives and to attain the advantages mentioned herein as well as those inherent in the disclosure. While presently preferred embodiments have been described herein, it will be understood that numerous changes may be made which will readily suggest themselves to those skilled in the art and which are accomplished within the spirit of the disclosure and claims.

What is claimed is:

1. A process for producing a cannabis product, the process comprises:
collecting a cannabis biomass having certain desirable qualities;
washing the cannabis biomass to create a washed cannabis biomass;
wet grinding the washed cannabis biomass to create a wet-ground cannabis biomass; and
drying the wet-ground cannabis biomass to create a dried cannabis biomass.

2. The process of claim 1 further comprising the step of dry grinding the dried cannabis biomass to create a powdered cannabis product.

3. The process of claim 1 wherein the cannabis biomass has a tetrahydrocannabinol (THC) content by weight of about 5% to about 50%.

4. The process of claim 1 wherein the cannabis biomass has a ratio of cannabidiol (CBD) to THC of about 1:1 to about 7:1.

5. The process of claim 1 wherein the cannabis biomass is soaked in water during the washing step for about 5 minutes to about 20 minutes.

6. The process of claim 5 wherein the water used to soak the cannabis biomass can be drained and fresh water can be added to the cannabis biomass and the cannabis biomass and water can be heated.

7. The process of claim 6 wherein the water and cannabis biomass are heated to about 185 degrees Fahrenheit to about 205 degrees Fahrenheit for about 10 minutes to about 20 minutes.

8. The process of claim 5 wherein the water is drained from the cannabis biomass, fresh water is added to the cannabis biomass and soaked therein for an additional 5 minutes to about 20 minutes.

9. The process of claim 8 wherein the water is drained from the cannabis biomass and the cannabis biomass is heated at about 215 degrees Fahrenheit to about 240 degrees Fahrenheit at a pressure of about 1 psi to about 15 psi above atmospheric pressure.

10. The process of claim 1 further comprises:
collecting black peppercorns;
soaking the black peppercorns in water for a predetermined amount of time; and
combining the soaked black peppercorns with the washed cannabis biomass.

11. The process of claim 10 wherein the black peppercorns are included with the cannabis biomass in an amount of about 2 percent to about 10 percent by weight of the cannabis bio mass used.

12. The process of claim 1 wherein the cannabis biomass is dried by letting the wet-ground cannabis biomass air dry.

13. The process of claim 1 wherein the cannabis biomass is dried via dehydration process at a temperature of about 122 degrees Fahrenheit to about 150 degrees Fahrenheit.

14. The process of claim 2 wherein the dried cannabis biomass is ground in intervals for certain amounts of time.

15. The process of claim 14 wherein each grinding interval lasts for up to about 45 seconds.

16. The process of claim 14 wherein the dried cannabis biomass is ground until the cannabis biomass product has a particle size in a range of from about 53 microns to about 125 microns.

17. The process of claim 16 further comprising separating out the cannabis biomass product with the desired particle size.

18. The process of claim 10 further comprising combining the black peppercorns and the washed cannabis biomass prior to the wet grinding step.
